(19) United States
(12) Patent Application Publication
(10) Pub. No.: US 2004/0214754 A1
(43) Pub. Date: Oct. 28, 2004

(54) Ophthalmic preparation containing glycomacropeptide

(76) Inventors: Edward J. Ellis, Lynnfield, MA (US); Charles D. Leahy, Concord, MA (US); Jeanne Y. Ellis, Lynnfield, MA (US)

Correspondence Address: CANTOR COLBURN, LLP, 55 GRIFFIN ROAD SOUTH, BLOOMFIELD, CT, 06002

(21) Appl. No.: 10/813,998

(22) Filed: Mar. 30, 2004

Related U.S. Application Data
(63) Continuation-in-part of application No. PCT/US02/31657, filed on Oct. 3, 2002.
(60) Provisional application No. 60/326,912, filed on Oct. 3, 2001.

(51) Int. Cl. A61K 38/16

(52) U.S. Cl. 514/8

(57) ABSTRACT

The present invention relates to an aqueous formulation to be instilled into the eye, or in which to pre soak or store an object to be inserted into the eye, such as a contact lens, an ointment, or a solid device to be inserted into the conjunctival sac. The preparations disclosed are utilized for the treatment of a tear film and ocular surface disorder known as keratoconjunctivitis sicca or dry eye syndrome. In general, the preparations of this invention are also effective for the relief of symptoms of eye irritation, such as those caused by dry environmental conditions or by contact lens wear. In accordance with the present invention, the ophthalmic preparation includes a glycomacropeptide component, similar to that found at the normal human ocular surface and in one exemplary and preferred embodiment, the glycomacropeptide is derived from mammalian milk, preferably bovine.

### CROSS-REFERENCE TO RELATED APPLICATION

This application is a continuation-in-part of International patent application No. PCT/US02/31657, filed Oct. 3, 2002, which claims benefit to U.S. provisional patent application No. 60/326,912, filed Oct. 3, 2001, both of which are hereby incorporated by reference in their entirety. Additionally, this application is related to pending U.S. patent application entitled, “OPHTHALMIC PREPARATION CONTAINING GLYCOPROTEIN”, filed by Applicant on Mar. 30, 2004, which is herein incorporated by reference in its entirety.

### STATEMENT REGARDING FEDERAL SPONSORSHIP

The U.S. Government has a paid-up license in this invention and the right in limited circumstances to require the patent owner to license others on reasonable terms as provided for by the terms of Grant Nos. 1 R43 EY12573-01, 2R44EY12573-02 and 5R44EY12573-03 awarded by the National Institute of Health.

### TECHNICAL FIELD

The present invention relates to ophthalmic preparations and more specifically relates to ophthalmic preparations for use as a tear film supplement, wherein the preparation includes at least one of a glycomacropeptide component.

### BACKGROUND OF RELATED ART

Initial descriptions and models of the tear film described the tear film as including three distinct layers and as being a three-layered, aqueous-dominated tear film. One of the layers comprises a mucin layer which serves primarily to render the hydrophobic ocular surface hydrophilic, so that the aqueous layer comprising the bulk of the tear film will spread evenly over the eye.

Current work in this field has shown that the classic aqueous-dominated tear film model has been replaced by the more probable concept of a mucin-dominated gel. This gel has its highest concentration of mucin at the epithelial surfaces of the cornea and conjunctiva, and the mucin concentration gradually decreases farther out into the tear film. In this model, the presence of mucin remains significant for the structure, stability and function of the entire tear film. Recent studies of the tear film using laser interferometry and confocal microscopy might be including the entire gel layer in indicating that the human tear film is 30 to 40 microns thick, more than four times thicker than earlier estimates.

Based on tear film physiology and clinical observations, tear film abnormalities are commonly designated by focus on a specific deficiency, such as an aqueous tear deficiency, kerato-conjunctivitis sicca (KCS), a mucin deficiency, a lipid abnormality, an impaired lid function, or an epitheliopathy. Although clinically useful, the simplistic concept of a lack of one component of the tear film as the cause of dry eye has given way to a much more sophisticated view of ocular surface disease that involves: (1) the health and regulation of the various glands contributing secretions to the tear film, (2) changes in the tear film itself, such as in osmolality and content of inflammatory mediators, and (3) what is viewed as a sort of “final common pathway”, the subsequent changes to the ocular surface. In fact, many clinicians and authors prefer the term “ocular surface disease” over “dry eye”, for it is change to the ocular surface, whatever the original cause, that results in the significant signs and symptoms of dry eye. The discomfort of ocular surface disease is expressed in ocular symptoms, such as dryness, grittiness, burning, soreness or scratchiness, with variation among individuals. These symptoms can also be exacerbated by factors such as environmental conditions, computer operation and contact lens wear. The combination of varying clinical signs and symptoms has also been termed dry eye syndrome.

Over the past twenty to thirty years many attempts have been made to provide an effective and long lasting treatment of dry eye symptoms, particularly for patients with moderate to severe KCS. These prior art attempts can be categorized on the basis of their physical state: ointments, emulsions, solid devices and aqueous based solutions or gels.

Ointments are generally anhydrous preparations based on mixtures of white petroleum and mineral oil. Because these formulations are greasy and cause blurred vision, they are not widely used other than in cases of severe symptoms, and are mostly limited to application at night just before sleeping. Emulsion based formulations for treating dry eye symptoms have emerged over the past ten years. One approach has been disclosed in a series of U.S. Pat. Nos.: 5,578,586; 5,371,108; 5,294,607; 5,278,151; 4,914,088, all of which are herein incorporated by reference in their entirety. These patents teach the methods and compositions for reducing evaporation of the aqueous layer from the surface of the eye. The method comprises applying an admixture of a charged phospholipid and a non-polar oil over the eye, preferably in the form of a finely divided oil-in-water emulsion. Another approach is described in U.S. Pat. Nos. 4,818,537 and 4,804,539, incorporated herein by reference in their entirety, where liposome compositions in the form of emulsions are claimed to provide enhanced retention on ocular surfaces and thereby alleviate the symptoms of dry eye.

Solid devices, in the form of ocular inserts, have been utilized for longer term symptomatic relief of dry eye. These devices are placed in the eye and slowly dissolve or erode to provide a thickened tear film. Often patients find these devices difficult to insert and once in place, they tend to be uncomfortable.

The most recommended and commercially successful methodology to treat dry eye symptoms is aqueous based solutions or gels. For the patient, eye drops are convenient and easy to apply relative to the other options mentioned above. There are at least thirty artificial tear products currently on the market from which to choose. For the most part the “active” ingredients in these present day artificial tear formulations are common water soluble or dispersable polymers such as: hydroxyethylcellulose; hydroxypropylmethylcellulose; methylcellulose; carboxymethylcellulose; polyvinyl alcohol; polyvinyl pyrrolidone; polyethylene glycol; carbomers; and poloxamers.

These currently marketed products, while providing temporary relief of symptoms—usually measured in minutes—are strictly palliative without long term effect. In fact, to truly maintain relief of symptoms in moderate to severe cases, an impractical schedule of doses would be necessary. With preserved solutions, the frequency of instillation can lead to signs and symptoms of irritation, making it necessary to utilize expensive and more cumbersome unit dose delivery packages.

The recent patent literature indicates a continued interest in pursuing synthetic based artificial tear solutions. For example, U.S. Pat. No. 5,460,834, incorporated herein by reference in its entirety, teaches the use of hydroxypropylmethylcellulose along with other ingredients as an ophthalmic solution, and U.S. Pat. No. 6,180,093 incorporated herein by reference in its entirety, discloses the use of polyvinylpyrrolidone in combination with other components to relieve eye dryness.

The art recognizes that an ophthalmic solution must provide an effective and long lasting treatment for symptoms of dry eye. One approach to achieving these aims is to provide a solution with tailored Theological properties, that is, a high viscosity solution that yields or flows under stress. Examples of this approach are disclosed in U.S. Pat. Nos. 5,075,104 and 5,209,927, incorporated herein by reference in their entirety, where the rheological properties of the ophthalmic solutions are attained through the use of carbomer polymers. These carbomer polymers have been found to be bio-adhesive as described in U.S. Pat. Nos. 5,225,196, 4,983,392 and 4,615,697, all of which are incorporated by reference in their entirety. It is believed that the bio-adhesive properties of the carbomer contributes to longer retention times in the eye. In fact, U.S. Pat. Nos. 5,075,104 and 5,209,927, incorporated by reference in their entirety, teach “that the carbomer polymers appear to function by maintaining or restoring the normal hydration equilibrium of the epithelial cells, thus protecting the cornea.

The search for useful ophthalmic solution polymers has extended into the area of bio polymers, with particular emphasis on the naturally occurring polysaccharides. One polymer, hyaluronic acid, and its sodium salt have received much attention over the past several years. In fact, one commercial product, Hylashield®, based on a high molecular weight sodium hyaluronate, has been successfully marketed as a dry eye treatment solution. The use of hyaluronic acid in artificial tear solution compositions is also taught in U.S. Pat. No. 5,460,834 which is incorporated by reference in their entirety. Other polysaccharides, such as carrageenan, tamarind gum and keratan sulfate have been claimed to have utility in artificial tear solutions as disclosed in U.S. Pat. Nos. 5,403,841 and 5,460,834, and 6,056,950, all of which are incorporated by reference in their entirety. In addition, polysaccharides, such as alginate, dextran, scleroglucan and xanthan have been used, or have been proposed for use in ophthalmic solutions.

The patent literature reveals one dated reference to the use of mucin in sterilized, preserved and stable solutions. U.S. Pat. No. 4,438,100, incorporated herein by reference in its entirety, describes mucin-containing solutions for application to sensitive mucous membranes of the oral cavity, the nasal system and the eye. The mucins utilized in this invention are non human mammalian mucins selected from the group consisting of buccal and gastrointestinal mucins. In fact, the source of their mucins is mucus, a mature and complex secretion containing a mixture of various mucin molecules as well as other proteins and associated contaminants of secretion. There is no distinction made between secreted mucins and mucins expressed by the surface cells of the oral cavity or gastrointestinal mucous membranes. Two very recent publications, U.S. Pat. No. 6,281,192 and U.S. Pat. No. 6,429,194, incorporated by reference in their entirety disclose ophthalmic applications of mucin derived from mammalian milk or milk byproducts: the mucin described was found to be a MUC1 type mucin similar to the transmembrane mucin expressed on the surface of the human eye.

### BRIEF SUMMARY OF THE INVENTION

The present application relates to ophthalmic preparations for use as a tear film supplement. More specifically, the invention relates to an ophthalmic formulation to be instilled into the eye, or in which to pre treat or store an object to be inserted into the eye, such as a contact lens or a solid device to be inserted into the conjunctival sac. The preparations disclosed are utilized for the treatment of disorders such as keratoconjunctivitis sicca or dry eye syndrome. In general, the preparations of this invention are also effective for the relief of symptoms of eye irritation, such as those caused by dry environmental conditions or by contact lens wear.

In particular, the present application relates to ophthalmic compositions including at least one milk-derived glycomacropeptide component, as well as to methods for their preparation and storage. The application also relates to a method of treating the eye by topically applying the composition of the present invention, when indicated, to provide lubrication and protection of the ocular surface, for the relief of dryness and discomfort symptoms, such as experienced in patients with dry eye and following traumatic injury or surgery, and when indicated to achieve the other effects mentioned above. In one preferred embodiment the compositions of the present invention are provided as buffered, sterile aqueous solutions. The subject compositions may be unpreserved (provided in a unit dose format) or may be preserved.

In one preferred embodiment, the glycomacropeptide is isolated from mammalian milk or milk byproducts. In another preferred embodiment the glycomacropeptide is isolated from bovine milk. In yet another preferred embodiment the glycomacropeptide is derived from dairy whey, a byproduct of cheese making. To form the ophthalmic preparations disclosed herein, other ingredients commonly employed in ophthalmic formulations are utilized to provide a balance of physiologically acceptable properties, depending on whether the final product is a solution, ointment, gel, lotion or solid. However, it will be understood that the glycomacropeptide can be derived from a number of other sources so long as the materials are suitable for the intended use described herein.

The above-discussed and other features and advantages of the present invention will be appreciated and understood by those skilled in the art from the following detailed description.

### DETAILED DESCRIPTION OF THE PREFERRED EMBODIMENTS

Glycroproteins are of great interest as components of mammalian membranes. Structurally they are composed of relatively short carbohydrate sequences covalently linked to a protein core. The glycoproteins of the cell surface appear to function in recognition, intercellular adhesion and modulation of certain intracellular transport event. That one characteristic phenotypic expression of tumorigenic transformation is an alteration in cell surface glycoproteins strongly implies an involvement of glycoproteins in at least several cell surface functions. They may enhance the production and stability of the tear film, and in particular may enhance the interaction of the aqueous tears and ocular surface glycoproteins, mucins and mucus. Physical activity and mechanisms of interaction of glycoproteins also contribute to protection of the surface. The carbohydrate groups of the protein are hydrophilic and have the ability to hold water and enhance the aqueous environment at the ocular epithelial cellular surface. The glycoproteins provide a physical mechanism of protection by their presence, bulk and hydration, as well as by binding pathogens and debris in their carbohydrate side chains.

Lipid globules in milk are enclosed in a membrane which is derived directly from the apical plasma membrane of mammary epithelial cells. This milk fat globule membrane (MFGM) can be readily obtained in quantity, and MFGM from bovine milk exhibits a higher degree of purity with respect to apical surface origin of the membrane material. Thus MFGM is an excellent source material for isolation of glycoproteins associated with the cell surface or, more properly, with a derivative of the apical cell surface. Over the past twenty years, there have been numerous publications elucidating both the isolation procedures for and the characterization of glycoproteins from milk.

With bovine MFGM, the presence of five to eight major glycoproteins have been detected with sodium dodecyl sulfate polyacrylamide gel electrophoresis (SDS-PAGE). The apparent molecular weight of these glycoproteins can range from about 30,000 to 40,000 daltons up to about 250,000 daltons. The more abundant glycoproteins have apparent molecular weights in the 30,000 to 40,000 dalton range. A mucoprotein has also been isolated from bovine milk and was found to have an apparent molecular weight of about 100,000 to 250,000 daltons.

Milk, both human and animal, natively contains an abundance of proteins including many glycoproteins. Dairy whey, a byproduct of cheese manufacturing, provides a plentiful and very inexpensive source of proteins. In fact, diary whey and products derived from dairy whey have long been recognized as food products, food additives and nutritional supplements.

The predominant protein in milk is casein, which comprises about 83% of the total milk protein. Bovine milk ranges from 2.5 to 3.2% casein by weight. The casein proteins can be divided into four major and one minor group with the properties indicated in Table 1.

TABLE 1
Distribution and properties of caseins.

| | | | SH per | S-S per | | |
|---|---|---|---|---|---|---|
| Component | % | P per mol | mol | mol | Mol Wt | pK |
| Casein | 75-85 | | | | | |
| aₛ₁ Casein | 45-55 | 8 | 0 | 0 | 23,500 | 4.6 |
| b-Casein | 25-35 | 5 | 0 | 0 | 24,000 | 5.0 |
| Kappa | 8-15 | 1 | 0 | 1 | 19,000 | 3.7-4.2 |
| Casein | | | | | | |
| Gamma | 3-7 | 1 | 0 | 0 | 20,000 | 5.8 |
| Casein | | | | | | |

The caseins are organized into conglomerates called micelles. These are roughly spherical and about 20-600 nm in diameter. The micelles are composed of sub-micelles of 8-20 nm diameter.

The caseins associate into sub-micelles through a combination of hydrophobic and electrostatic forces. Alph-S1 casein has three distinct regions: strongly hydrophobic; highly negatively charged; and neutral.

It has clusters of phospho-seryl residues that are highly charged at pH 6.6.

Alpha-S2 casein is the most hydrophilic casein. Its C-terminal is positively charged and thus electrostatic interactions may be important for its stability.

Beta casein is the most hydrophobic of the caseins but has at least one Icuster of phospho-seryl residues.

Kappa casein is the smallest casein but plays the most important role in stabilizing the micelles. It has tetrasaccharide chains attached to it and at pH 6.6 the C-terminal has a net negative charge of −16 or −17. This macropeptide end of the molecule is believed to be on the surface. It is the most structured of the caseins, with a significant proportion of alpha helix and beta sheet.

Kappa casein consists of 169 amino acids and has a molecular weight of 19,000. The protein can contain from 0 to 5 trisaccharide units composed of a N-acetylneuraminyl (2→6) b-galactosyl(1→3 or 6) N-acetylgalactosamine. It contains one serine phosphate group and two cysteine residues and there are two genetic variants of kappa casein. The average hydrophobicity of k-casein is 1205. Unlike a s and b -caseins, k-casein has charged sections at both ends of the molecule. The 53 C-terminal amino acids have a net charge of −11, contain the 1 phosphate group and all of the carbohydrate associated with k-casein. This carbohydrate further increases the net negative charge of this portion of the molecule. The remainder of the molecule is very hydrophobic and contains net positive charge at pH 6.7.

Most of the molecules net charge is not derived from serine phosphate groups and thus k-casein is soluble in the presence of Ca⁺⁺. It can also interact with a s and b -caseins and stabilize them in the presence of calcium ions. Extensive studies with model systems have demonstrated that while the complex is stable to the presence of Ca⁺⁺. In fact, Ca⁺⁺ must be present for the complex to form.

Casein micelles give milk most of its white appearance. They are held together by colloidal calcium phosphate in which calcium is associated with phosphate, magnesium and citrate. Removal of the calcium (e.g., by chelation) causes dissociation of the micelles into sub-micelles. The micelles are highly stable to heat and also depend on the forces between the caseins for stability. The hydrophobic region of the kappa casein acts as the main stabilizer with the charged macropeptide end projecting from the micelle surface. Thus the outside of the micelles are negatively charged and repel each other. This accounts for the fact that the micelles remain in suspension in normal gravity.

The behavior of the micelles under the action of acid proteases is exploited in the manufacture of cheese. Acid proteases such as the chymosin of rennet hydrolyse only one bond in kappa-casein at pH 6.6-6.7. This is the peptide bond between residues 105 and 106 (phenylalanine and methionine) and releases the charged macropeptide into solution. The remainder of the kappa-casein, para-kappa-casein, stays in the micelle. With the charged protection removed, the micelles begin to aggregate through hydrophobic interactions, growing from short chains to major clusters. This is visible as the coagulation of milk brought about by the enzyme. As time progresses, the casein aggregates rearrange and become denser, eliminating water (syneresis of the whey).

Whey, a byproduct in cheese production, is a valuable source of glycoproteins and glycomacropeptide. Glycomacropeptide (GMP) is a casein-derived whey peptide. This casein macropeptide is found in “sweet” whey, but not in “acid” whey. When milk is treated with chymosin during cheesemaking, the milk protein (k-casein) is hydrolyzed into two peptides. The larger peptide containing amino acid residues 1-105 is called para-k-casein, and becomes part of the cheese curd, while the smaller peptide containing amino acid residues 106-169, the GMP, becomes soluble and part of the whey. The peptide is relatively small, with a molecular weight of 8,000 Daltons. There are two major variants of GMP, variant A and variant B, that differ in two amino acids. Different abbreviations are used to identify GMP, but all refer to the same molecule found in whey. CMP is the abbreviation for caseinmacropeptide, while CGMP is used as a more descriptive designation of casein-glycomacropeptide. It is sometimes also referred as CDP (casein-derived peptide) or CGP (caseinglycopeptide) to identify its origin.

Glycomacropeptide (GMP) has many unique characteristics when compared to other whey proteins. The “glyco” portion of glycomacropeptide refers to the saccharide groups that are attached to the peptide backbone of the molecule. Glycomacropeptide contains various amounts of covalently attached oligosaccharides, including N-acetylneuraminic acid (sialic acids), galactose and N-acetyl-galactosamine. The most prominent of theses is sialic acid, which comprises about 7-8% of the glycomacropeptide.

The biological function of the k-casein glycomacropeptide can be attributed to both the carbohydrate and the peptide make up of the k-casein. The two most important carbohydrate components are N-acetylneuraminic acid (NANA) and N-acetylgalactosamine. NANA has been shown to have a large number of biological functions, which include: regulation of shell shape and lifetime, component of hormones, contribution to the binding of calcium, contribution to regulation of cell growth and differentiation, interaction with antibodies, component of cell receptors, protection against bacteria adhesion and contribution in interactions with viruses. The manner in which the carbohydrate moieties influence the biological function of GMP still requires further clarification, since specific peptides derived from trypsin and chymotrypsin action on GMP have varied biological function.

The biological activity of the GMP is thought to depend on the content and structure of the sugar moieties. Inhibition of splenocyte proliferation, toxin binding, inhibition of hemagglutination, stimulation of bifidobacteria have all been related to the NANA component of the GMP. The mechanism is thought to be that the NANA interacts with specific receptors.

Most of the studies of the biological activity of GMP have been done in vitro, and require in vivo confirmation. In some cases, such as inhibition of bacterial adhesion or regulator activity in the digestive tract, the GMP can act without being adsorbed. Other actions, such as immunomodulation or interactions with blood components would be expected to require absorption. This would occur following the action of digestive enzymes with the formation of smaller fragments with or without associated carbohydrates. Intact macropeptides have been found in the plasma of new-borns fed cow's milk-based formula.

Recovery and purification of glycomacropeptide can be carried out utilizing standard methods known in the art. These would include, but are not limited to, membrane filtration and microfiltration, tangential flow filtration, chromatography (e.g., size exclusion, ion exchange, affinity), extraction, adsorption, precipitation (with non-solvents, salts, etc.), density gradient fractionation, electrophoresis, electrodialysis, isoelectric focusing, acid or base hydrolysis, and chymosin hydrolysis.

The glycomacropeptide of this invention are mucin-like substances that can vary in molecular weight from a few thousand to one or two hundred thousand. The glycomacropeptide of this invention contain oligosaccharides to the extent of about 4% to about 15%. The glycomacropeptide of this invention are robust and can be autoclaved without significant degradation or chemical modification.

The scientific literature reveals a number of techniques for characterizing the various types of glycomacropeptide. These techniques include, but are not limited to, chromatographic techniques or one or two dimensional gel electrophoresis, particularly SDS-PAGE, followed by direct protein staining (e.g., silver staining) or immunohistochemical staining (e.g., Western blotting or Northern blotting) and image analysis, immunoprecipitation techniques, amino acid analysis, carbohydrate determination, lectin binding probes, light scattering, scanning electron microscopy, mass spectrometry, peptide sequencing, MALDI, protein nitrogen content and ash.

Although not being held to any one theory we believe that the glycomacropeptide described in this invention act to protect and lubricate the ocular surface, as in the role of the natural surface glycoproteins and mucins, which are expressed by the entire surface epithelium of the conjunctiva and cornea. By supplementing the natural epithelial surface glycoproteins, the lubrication and protection of the ocular surface is enhanced, in order to slow the progression, and associated development of symptoms, of changes to the ocular surface epithelium, such as decreased tear film stability, increased staining with fluorescein sodium or rose bengal, decreased goblet cell density and the development of squamous metaplasia seen with ocular surface disease. The property of viscosity in the preferred embodiment is primarily targeted to assist in retention of the invention in the eye at the ocular surface, as well as for lubrication and comfort associated with instillation. Viscosity is not the physical property which gives the glycomacropeptide formulation of this invention its “mucomimetic” function. This invention primarily protects and lubricates the ocular surface and interacts with the gel-forming secreted mucins of the tear film, thereby enhancing the spreading of the tear film, and by default of instillation adds to the tear film volume and hydration of the ocular surface. The “mucomimetic” effects of this invention protect the ocular surface from dryness and absorb shear forces of the blink, and assist the eye's own secreted gel forming mucins (predominantly MUC5) in maintaining their viscoelastic properties and ensuing structure and stability of the tear film, thereby slowing or preventing the changes to the ocular surface seen in dry eye conditions.

The amount of glycomacropeptide in an ophthalmic formulation can vary greatly depending on the product type. For example, in contact lens related solutions the glycomacropeptide concentration would vary from about 0.0001% to 5.0% by weight. In dry eye preparations the glycomacropeptide level could vary from about 0.1% to about 10.0% by weight. In a solid ocular insert delivery device the glycomacropeptide level could range to about 90% or greater by weight. Within each type of preparation the concentration might be varied, depending on such factors as the severity of the dry eye condition being treated, to enhance particular properties of the glycomacropeptide solution. These ranges are for the purpose of illustration and are not meant in any manner to limit the scope of the claims.

Exemplary ophthalmic compositions include a glycomacropeptide from any number of the exemplary sources described herein before. In addition, other formulation components may be employed as required:

### VISCOSIFIERS

Cellulose derivatives are commonly used to increase viscosity. Specific cellulose derivatives include: hydroxypropylmethylcellulose, carboxymethylcellulose, methylcellulose, hydroxyethylcellulose, etc. Some polysaccharides may also be utilized to increase the viscosity of ophthalmic solutions and include xanthan, scleroglucan, carrageenans, tragacanth gum, hyaluronic acid etc. Other viscosifiers that are useful include polyvinylpyrrolidone, polyvinyl alcohol, polyethyleneoxide, polyacrylic acid and crosslinked polyacrylic acid. Generally, viscosifiers are present in the amount of 0.1 to 0.75% by weight of the solution.

### BUFFERING AGENTS

Any pharmaceutically acceptable buffer system may be utilized and include phosphates, borates, citrates, acetates and carbonates in amounts necessary to produce a pH of about 6.0 to about 8.0.

### TONICITY AGENTS

The tonicity of the ophthalmic solutions described here can be adjusted to either hypotonic, isotonic or hypertonic relative to normal tears by use of generally used materials know to the art. Sodium and potassium chloride are widely used to adjust tonicity. Other agents include dextrose, mannitol, sorbitol and urea.

### HUMECTANTS

Water binding compounds aid in retaining moisture on the ocular surface and include glycerin, propylene glycol, polyethylene glycol.

### WETTING AGENTS

Certain compounds are useful to promote surface wetting, whether it be the ocular surface or the surface of a contact lens. One category that is preferred is the polyoxamers. These polyethyleneoxide-polypropyleneoxide-polyethyleneoxide block copolymer are available from BASF. Other compounds include the Tetronics®, reverse Pluronics® and the reverse Tetronics®, also available from BASF.

### PRESERVATIVES

The compositions of this invention may include a preservative in an effective amount. Preservatives known to the art include alkyldimethyl benzylammonium chloride (BAK), chlorhexidene gluconate (CHG), polyhexamethylene biguanide (PHMB), other polyquats and sorbic acid. The subject compositions may also include a co-preservative and/or chelating agent, such as ethylenediaminetetraacetic acid (EDTA) and its salts.

### OTHER ADDITIVES

In some cases it may be beneficial to include other components in an ophthalmic solution. These include specific ions, such as Ca++, Zn++ and Mg++, Cu++, selenium, vitamins, such A, C and E, to promote ocular health. The compositions described in this invention may also be utilized as vehicles for drug delivery. Drugs often used in the eye include anti-glaucoma compounds, anti-inflammatory agents and anti-infective agents.

As previously described, this invention finds particular utility as lubricating eye drops, i.e., an artificial tear solution, a tear fluid supplement, a delivery vehicle for topical ophthalmic drug application. In most of these applications, the compositions of this invention are provided in a buffered, sterile aqueous solution. Typically, these solutions have a viscosity from about 1 to 100 cps. As a solution the compositions of this invention are dispensed in the eye in the form of an eye drop. It should be understood, however, that the compositions described in this invention may also be formulated as viscous liquids, i.e., viscosities from several hundred to several thousand cps, gels or ointments. In these applications the glycomacropeptide component could be ,for example, dispersed or dissolved in an appropriate vehicle such as Lubragel, GRR Lubricating Jelly or Karajel, all trademarked products of United-Guardian, Inc., Hauppauge, N.Y.

The compositions of this invention may also be formulated as solid ocular inserts that dissolve or erode over time when placed in the cul-de-sac of the eye.

Swelling-controlled release devices would consist of glycomacropeptide homogeneously dispersed in a polymer such as a water soluble cellulosic. When the insert is placed in the eye, the tear fluid begins to penetrate the matrix, followed by swelling, and finally dissolution, of the matrix. As this process occurs, glycomacropeptide is released into the eye to provide relief of dry eye symptoms over a long period of time.

Erodible devices would again consist of glycomacropeptide homogeneously dispersed in a polymer matrix. In this case, glycomacropeptide is released by a chemical reaction (hydrolysis) that results in solubilization of the matrix polymer, usually at the surface of the device. Generally, the matrix material is a polyanhydride, poly(ortho ester), polylactic acid or a polyglycolic acid.

In another embodiment the glycomacropeptide may be chemically modified or crosslinked to act as its own “matrix”, where the glycomacropeptide comprises the entire, or nearly entire, device, thus providing the maximum amount of glycomacropeptide available to the eye.

Furthermore, in some contact lens related embodiments, the glycomacropeptide disclosed herein may be incorporated into contact lens soaking and conditioning solutions as well as lubricating eye drops for contact lens wearers.

In another embodiment the glycomacropeptide may be utilized in drug delivery. The most common and convenient method for delivery of ocular drugs is by way of topical eye drops. Generally, the solution vehicles employed are quickly diluted by the tear fluid and drain from the eye in a matter of minutes. This short residence time hinders the absorption and hence the bioavailability of the drug in the eye. Oftentimes the short residence time is overcome by greatly increasing the concentration of the drug to improve bioavailability. This often leads to significant undesirable side effects due to the systemic actions of many of the ocular drugs currently prescribed.

Much research has been done to improve the residence time of the drug vehicle at the ocular surface and also to promote interaction or association of the drug with the vehicle. One approach that has been commercialized is to utilize a crosslinked carboxy-functional polymer such as Carbopol®, supplied by B. F. Goodrich. The bioadhesive nature of this polymer has been the basis for controlled release ophthalmic formulations as described in U.S. Pat. No. 4,615,697 and U.S. Pat. No. 5,188,826, both of which are incorporated by reference in their entirety. These crosslinked carboxy-functional polymers swell in aqueous solution but remain as micron-size hydrated particles. Furthermore, at neutral pH, they are substantially anionic in nature. Since many ophthalmic drugs, for example timolol and pilocarpine, are positively charged, they will associate with the negatively charged polymer particles through electrostatic interaction. Also, since the hydrated particles are microporous, the drug can be absorbed into the matrix. When an ophthalmic solution of this type is placed in the eye, the hydrated polymer particles adhere to the mucosal surface, providing extended residency time. During this residence the drug is released from the hydrated polymer particles, thus providing for a more efficient local delivery to the eye.

The glycomacropeptide of the present invention are considered “bioadhesive” given their functions in association with the plasma membrane of mammary epithelial cells. Given this information one would expect the glycomacropeptide of this invention to act in a similar manner to the crosslinked carboxy-functional polymers as an ophthalmic drug delivery vehicle. In practice, the glycomacropeptide of this invention provide superior retention time due to their ability to interact not only with the epithelial surface but also with the natural mucins in the tear film.

The present invention provides an ophthalmic preparation for the treatment of an ocular condition known as dry eye. As such, the present invention may be described in certain embodiments as a method of treating dry eye disorders in a mammal comprising administering to said mammal an amount of glycomacropeptide compound effective to provide a therapeutic effect to said mammal. An aspect of the present invention is also a method of providing continued therapy to a mammal by administrating in a prescribed, regular basis to said mammal. In certain preferred embodiments of the invention, a mammal or patient to receive the glycomacropeptide compound may be a human or animal. Furthermore, a glycomacropeptide compound as used in the practice of the disclosed ophthalmic preparations and methods is glycomacropeptide that is derived from milk or milk byproducts. As disclosed herein and as used in the preparations and methods of the present invention, a glycomacropeptide compound may be formulated into a solution, ointment, gel, lotion or solid.

Effective dosages as described herein include, but are not limited to, an amount of glycomacropeptide compound from about 0.01 mg to about 5 mg per dose when delivered in the form of an ophthalmic solution. When the ophthalmic vehicle is a gel or ointment the amount of glycomacropeptide delivered can range up to about 20 mg per dose. If the glycomacropeptide is in the form of an ocular insert the amount of glycomacropeptide introduced into the eye can be up to about 150 mg. It should be noted that in the case of an ocular insert the glycomacropeptide is delivered by continuous release over a prolonged period of time. It is well known in the pharmaceutical art to prescribe drugs based on whether the patient is a human or animal and based on the type and severity of the disease. The ophthalmic preparations of this invention are well within the skill of a practitioner in the art. In an alternate method of describing an effective dose, an effective amount may be described, in certain embodiments as an amount that is effective to achieve a reduction in the signs and/or symptoms of dry eye in a patient.

Preferred formulations include ophthalmic compositions in which a glycomacropeptide is formulated as an ocular insert. More preferably, the glycomacropeptide is formulated as an ointment, lotion or gel. Most preferably, the glycomacropeptide is formulated as an ophthalmic solution.

In certain aspects, the present invention includes pharmaceutical compositions in both unit dose form and in multi-dose form. These compositions are utilized to treat dry eye, which comprises: an amount of glycomacropeptide such that one or more doses thereof are effective to stabilize or lessen the signs and symptoms of dry eye of said patient upon periodic administration

Also in certain aspects, the present invention includes veterinary compositions in both unit dose form or in multi-dose form. These compositions are utilized to treat dry eye in an animal, which comprises: an amount of a glycomacropeptide such that one or more doses thereof are effective to stabilize or lessen the signs and symptoms of dry eye of said animal upon periodic administration

An aspect of the present invention may also be described as a therapeutic package for dispensing to, or for use in dispensing to, a mammal being treated for dry eye syndrome comprising: one or more dosages, each dose delivered from a unit dose container or from a multi-dose container. The dosage form contains the glycomacropeptide of this invention, such that said one or more doses thereof are effective to stabilize or lessen the signs and symptoms of dry eye of said mammal upon periodic administration and the doses being administered periodically, and a finished pharmaceutical container or package therefor, said container containing said unit doses or multi-doses and labeling directing the use of said package in the treatment of said mammal.

The ophthalmic therapeutic preparations of this invention, in the form of a solution, lotion, ointment or gel, can be packaged in unit doses or multi-dose containers. The patient would utilize the packaged product in accordance with the prescribed regimen. Typically, in the case of an ophthalmic solution product, the patient will instill one or more drops into the eye as prescribed and/or as needed. The product container and associated packaging will bear identification, information and instructions in accordance with local, federal and foreign governmental regulations. The inclusion of a “package insert” is also generally required. The “package insert” will provide information pertaining to contents, action, indications, contraindications, warnings, how supplied, safety information and precautions, as well as directions for use.

The following examples are presented for purposes of illustrating the practice of the invention and are not intended as limitations on the scope thereof.

### EXAMPLE 1

Glycomacropeptide (GMP), also known as caseinmacropeptide (CMP), casein-glycomacropeptide (CGMP), casein-derived peptide (CDP) and caseinoglycopeptide (CGP) all refer to the same glycopeptide. This material can be obtained from commercial sources in the form of a nutritional product or supplement and also can be obtained from Sigma-Aldrich, catalog number C-7278.

### EXAMPLE 2

Glycomacropeptide (GMP) contains 64 amino acid residues and the amino acid profile is as follows:

| | Grams per |
|---|---|
| Amino Acid | 100 g powder |
| Alanine | 5.0 |
| Arginine | 0.4 |
| Aspartic Acid | 7.2 |
| Cystine | 0.1 |
| Glutamic Acid | 17.0 |
| Glycine | 0.9 |
| Histidine | 0.2 |
| Isoleucine | 8.3 |
| Leucine | 2.3 |
| Lysine | 5.0 |
| Methionine | 1.6 |
| Phenylalanine | 0.4 |
| Proline | 9.3 |
| Serine | 5.0 |
| Threonine | 11.3 |
| Tyrosine | 0.5 |
| Valine | 6.7 |
| Tryptophan | 0.08 |

### EXAMPLE 3

Glycomacropeptide (GMP) can be obtained in commercial quantities from dairy related industries. The following is a list of typical properties of a commercially available GMP.

| Property | Typical Range | Test Method |
|---|---|---|
| GMP Purity (%) | 97 ± 1 | Electrophoresis (SDS-PAGE) |
| Moisture (%) | 6.0 ± 0.2 | Vacuum Oven |
| Total Nitrogen (%) | 12.2 ± 0.2 | Leco Combustion |
| GMP (N × 6.94) (%) | 85.0 ± 1.5 | Calculated |
| Fat (%) | 0.6 ± 0.2 | Mojonnier |
| Ash (%) | 6.3 ± 0.2 | Gravimetric |
| Lactose (%) | <1.0 | Enzymatic Assay |
| pH | 6.4 ± 0.2 | 10% Sol. @20° C. |
| Foreign Matter | None | Visual |

### EXAMPLE 4

Glycomacropeptide is a protein segment containing 64 amino acid residues with pendent oligosaccharide groups. The SDS-Polyacrylamide gel electrophoresis (SDS-PAGE) pattern is obtained with a 4-15% tris-HCl gel and coomassie blue staining. Appropriate molecular weight standards are utilized as calibration standards. The apparent molecular weight of glycomacropeptide by SDS-PAGE is approximately 31,000 daltons.

### EXAMPLE 5

The following example illustrates the use of the glycomacropeptide (GMP) of this invention as the active ingredients in ophthalmic solutions for the treatment of dry eye signs and symptoms. The formulations listed below:

| | AMOUNT IN | AMOUNT IN |
|---|---|---|
| | GRAMS | GRAMS |
| INGREDIENTS | A | B |
| Glycomacropeptide (GMP) | 1.00 | 2.00 |
| Hydroxyethylcellulose | 0.4 | 0.40 |
| Natrosol 250M Pharm | | |
| Sodium chloride | 0.48 | 0.48 |
| Sodium borate decahydrate | 0.12 | 0.12 |
| Boric acid | 0.74 | 0.74 |
| Water | 97.21 | 96.21 |

were prepared in the following manner: Weigh out the water and add to an appropriate beaker equipped with a stirring apparatus. With stirring, add the sodium chloride, sodium borate decahydrate and boric acid to the water. Stir until the salts have completely dissolved, about 10 minutes. With vigorous stirring, add the GMP slowly to the batch. Continue vigorous stirring until the GMP is finely dispersed, about 30 minutes. With moderate stirring, add the hydroxyethylcellulose slowly to the batch. Continue stirring at a moderate rate for 2 hours. Allow the batch to deaerate for 15 minutes. Place batch in a suitable sealed container for autoclaving. Autoclave the batch for 60 minutes at 121° C. Remove solution container from the autoclave. Open container and perform batch testing. Once prepared, the formulations listed above were tested and the following solution properties were determined.

| | VALUE | VALUE |
|---|---|---|
| PROPERTY | A | B |
| Viscosity, cps | 12.5 | 14.5 |
| Osmolality, mOsm/kg | 305 | 309 |
| pH | 7.3 | 7.3 |
| Appearance | clear | clear |
| Color | colorless | colorless |

### EXAMPLE 6

The following example illustrates the use of glycomacropeptide (GMP) in a preserved ophthalmic solution. The formulation described below was prepared by the detailed process given in Example 5.

| | AMOUNT IN |
|---|---|
| INGREDIENT | GRAMS |
| Glycomacropeptide (GMP) | 1.00 |
| Hydroxyethylcellulose | 0.50 |
| Natrosol 250M Pharm | |
| Propylene glycol | 0.50 |
| Sodium chloride | 0.20 |
| Sodium borate decahydrate | 0.12 |
| Boric acid | 0.70 |
| Potassium sorbate | 0.15 |
| Edetate disodium | 0.05 |
| Water | 96.78 |

The finished solution was tested and the following physical properties were generated.

| PROPERTY | VALUE |
|---|---|
| Viscosity, cps | 26.5 |
| Osmolality, mOsm/kg | 300 |
| pH | 7.1 |
| Appearance | Clear |
| Color | colorless |

### EXAMPLE 7

This example illustrates the use of the glycomacropeptide (GMP) of this invention in contact lens solutions. The contact lens solution base utilized in this example was RENU™ MultiPlus, manufactured and sold by Bausch & Lomb. RENU™ is a multi-purpose solution for the storage and care of soft hydrogel contact lens. The following formulation was prepared by adding 0.5% by weight of glycomacropeptide to RENU™.

| INGREDIENT | AMOUNT IN GRAMS |
|---|---|
| glycomacropeptide | 0.25 |
| RENU ™ Lot CH7058 | 49.75 |

The glycomacropeptide was thoroughly dispersed in the RENU™ by vigorous stirring for one hour. The resulting solution was clear with a pH of 7.1 and an osmolality of 294 mOsm/kg. It is expected that the addition of glycomacropeptide to a soft contact lens solution will result in improved lubricity and comfort to the lens wearer.

### EXAMPLE 8

The following example illustrates the use of glycomacropeptide (GMP) in an ophthalmic gel for the treatment of dry eye signs and symptoms. Lubragel® MS, available from United-Guardian, Inc. was chosen as the gel base. Lubragel® MS is composed of polyglycerolmethacrylate and propylene glycol preserved with parabens. A 2% glycomacropeptide (GMP) in Lubragel® MS was prepared by thoroughly mixing the Milcin® into the gel base to form a uniform dispersion. The resulting gel was slightly hazy.

### EXAMPLE 9

The following example illustrates the use of glycomacropeptide (GMP) as an erodible ocular insert to provide long lasting treatment of dry eye. Approximately 50 mg of glycomacropeptide (GMP) was placed in a micro KBr press. The press was heated to about 80° C. and then tightened to compress the GMP under heat and pressure. The KBr press was allowed to cool to room temperature and the sample was removed. The GMP was in the form of a solid disk about 5 mm in diameter and about 2 mm in thickness. The GMP disk was placed in water and slowly eroded over about two hours.

### EXAMPLE 10

This example illustrates the use of the glycomacropeptide (GMP) of this invention as a component in an allergy relief solution. The particular ingredient for allergy relief chosen was olopatadine hydrochloride. Patanol® is a commercially available solution containing 0.1% by weight of olopatadine. The other solution components are sodium chloride, a phosphate buffer system and benzalkonium chloride as a preservative. Patanol® is an isotonic solution with a pH of about 7. A solution was prepared by adding 1.0% by weight of GMP into Patanol® solution which is manufactured and sold by Alcon Pharmaceuticals. The GMP was compatible in the Patanol® solution and is expected to provide improved lubricity and comfort to the patient.

### EXAMPLE 11

This example illustrates the use of the glycomacropeptide (GMP) of this invention in an antibacterial ophthalmic solution with activity against a broad spectrum of gram-postive and gram-negative ocular pathogens. The antibacterial agent chosen was ciprofloxacin hydrochloride. Ciloxan® is a commercially available solution containing 0.3% ciprofloxacin. The other solution ingredients are sodium acetate, mannitol, edetate disodium and benzalkonium chloride. Ciloxan® is an isotonic solution with a pH about 4.5 that is manufactured and sold by Alcon Pharmaceuticals. A solution was prepared by adding 1.0% by weight of GMP into Ciloxan® solution. The GMP was compatible in the Ciloxan® solution and is expected to provide improved lubricity and comfort to the patient.

### EXAMPLE 12

This example illustrates the use of the glycomacropeptide (GMP) of this invention in an antibiotic and steroid combination ophthalmic solution. The antibiotic agent chosen was tobramycin and the steroid chosen was dexamethasone. Tobradex® is a commercially available solution containing 0.3% by weight tobramycin and 0.1% by weight of dexamethasone. The other solution ingredients are hydroxethylcellulose, sodium chloride, sodium sulfate, Tyloxapol®, edetate disodium and benzylalkonium chloride as the preservative. Tobradex® is manufactured and sold by Alcon Pharmaceuticals. A solution was prepared by adding 1.0% by weight of GMP into Tobradex® 4 solution. The GMP is expected to provide improved lubricity and comfort to the patient.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing form the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. An ophthalmic preparation comprising a glycomacropeptide derived from one of mammalian milk and a milk byproduct.

2. An ophthalmic preparation in accordance with claim 1 wherein the glycomacropeptide component is derived from dairy whey.

3. An ophthalmic preparation in accordance with claim 1 wherein the glycomacropeptide is derived from casein.

4. An ophthalmic preparation in accordance with claim 3 wherein the glycomacropeptide containing component has an apparent molecular weight of about 31,000 daltons

5. An ophthalmic preparation in accordance with claim 3 wherein the glycomacropeptide containing components have a carbohydrate content of about 3% by weight to about 50% by weight.

6. An ophthalmic preparation in accordance with claim 3 wherein said preparation is in the form of one of a solution, an ointment and an ocular insert.

7. An ophthalmic preparation in accordance with claim 3 wherein the glycomacropeptide is present in an amount from about 0.001% to about 10.0% by weight.

8. An ophthalmic preparation in accordance with claim 3 wherein the glycomacropeptide is present in an amount from about 10% to about 90% by weight.

9. An ophthalmic preparation in accordance with claim 1 wherein the glycomacropeptide is autoclavable.

10. An ophthalmic preparation in accordance with claim 1 further comprising a material selected from the group consisting of a buffering agent; a viscosity modifying agent; a tonicity modifying agent; a humectant compound; and a therapeutic drug.

11. An ophthalmic preparation in accordance with claim 3 wherein said glycomacropeptide is derived from sweet whey.

12. An ophthalmic preparation in accordance with claim 11, wherein said glycomacropeptide is derived from purified whey.

13. A method of treating dry eye in a mammal comprising administering an ophthalmic preparation to said mammal in need thereof an amount of a glycomacropeptide component that is contained therein and that is effective to treat the dry eye of said mammal.

14. The method of claim 13, wherein said effective amount of the glycomacropeptide component is from about 0.01 mg to about 5.0 mg per dose.

15. The method of claim 13, wherein said effective amount of the glycomacropeptide component is from about 5.0 mg to about 20.0 mg per dose.

16. The method of claim 13, wherein said effective amount of the glycomacropeptide component is from about 20.0 mg to about 200 mg per dose.

17. A therapeutic package for dispensing to, or for use in dispensing to, a patient being treated for dry eye comprising:
one or more unit doses, each such unit dose comprising an amount of glycomacropeptide therein such that periodic administration of one or more of said unit doses is effective to treat said dry eye condition, and
a finished pharmaceutical container therefore, said container containing said unit dose or multiple doses, said container further including labeling;
said labeling directing the use of said package in the treatment of said dry eye condition in a dosage regimen under which the delivery of said glycomacropeptide is confined to the period during the day proximate to the time of day at which the patient requires treatment, and further directing the use of said package in conjunction with the concomitant administration to said patient of one or more unit doses providing a therapeutically effective amount of glycomacropeptide to said patient.

18. A package according to claim 17 in which the delivery of said glycomacropeptide is directed to be confined proximate to the time of waking and the delivery of said glycomacropeptide is confined proximate to the time of onset of sleeping.

19. A therapeutic package for dispensing to, or for use in dispensing to, a patient being treated for dry eye comprising:
one or more unit doses, each such unit dose comprising an amount of glycomacropeptide therein such that periodic administration of one or more of said unit doses is effective to treat said dry eye condition, and
a finished pharmaceutical container therefore, said container containing said unit dose or multiple doses, said container further containing or comprising labeling;
said labeling directing the use of said package in the treatment of said dry eye condition in a dosage regimen under which the delivery of said glycomacropeptide is one or more times daily, or as directed by a physician.

20. A package according to claim 19 wherein said labeling directs the use of said package in the treatment of dry eye induced by an activity selected from the group consisting of: contact lens wear and prolonged viewing of a computer screen.

21. A package according to claim 19 that is produced by form, fill and seal manufacturing wherein each container contains from about 0.50 ml to about 1.50 ml of glycomacropeptide solution.

22. A package according to claim 19 comprising a bottle, dropper tip and cap wherein each bottle contains from about 2.0 ml to about 30.0 ml of glycomacropeptide solution.

23. A package according to claim 21 in which said unit dose containers are provided to accommodate an uninterrupted dosage regimen basis of at least one month.
